(19) United States
(12) Patent Application Publication
(10) Pub. No.: US 2018/0218888 A9
(43) Pub. Date: Aug. 2, 2018

(54) PLASMA ETCHING APPARATUS

(71) Applicant: SPTS TECHNOLOGIES LIMITED, Newport (GB)

(72) Inventor: MAXIME VARVARA, COPPONEX (FR)

(73) Assignee: SPTS TECHNOLOGIES LIMITED, Newport (GB)

(21) Appl. No.: 14/285,727

(22) Filed: May 23, 2014

(30) Foreign Application Priority Data
Oct. 15, 2013 (GB) 1318249.8

(51) Int. Cl. H01J 37/32; F16B 7/04

(52) U.S. Cl. CPC H01J 37/32807; H01J 2237/334; H01J 37/32357; F16B 7/0406

(57) ABSTRACT

A plasma etching apparatus includes first, second and third chambers, and a plasma generation device. An inner cross-sectional area and shape of the second chamber interior substantially corresponds to the upper surface of a substrate, and a substrate support is disposed so that, in use, the substrate is substantially in register with the interior of the second chamber, and the upper surface of the substrate is positioned at a distance of 80 mm or less from the interface between the second and third chambers.

### CROSS-REFERENCE TO RELATED APPLICATIONS

A claim of priority is made to UK Patent Application No. 1318249.8 filed on 15 Oct. 2013, the disclosure of which is incorporated herein in its entirety.

### BACKGROUND

This invention relates to a plasma etching apparatus and to an associated kit of parts and a method of plasma etching a substrate.

Processes for the manufacture of devices on semiconductor wafers include a large number of plasma etch process steps which are carried out in a variety of plasma etch tools. Typically, plasma etch processes are used to selectively remove material from the areas of the wafer that are not covered by a mask. Etch depths can vary from nanometres to hundreds of microns in a variety of materials, such as silicon, GaAs, aluminium, and silicon dioxide. In all plasma etch processes there is a general requirement to provide a uniform, repeatable etch process. These qualities should be apparent both within a die (over an area of a few square millimetres or square centimetres) and across the entire wafer (currently to a diameter of 300 millimetres, although still large diameters may become the commercial norm in the future).

It is of course economically beneficial to be able to etch features as rapidly as possible due to the relatively high cost of plasma etch tools and clean rooms, and also because clean room floor space is at a premium, and therefore efficient utilisation of clean room space is important. Unfortunately, in practice in most cases a reduction in process times, through the use of high etch rates, results in a less uniform process performance. As such, it is typical for a compromise to be made so that both uniformity and etch rate are at acceptable levels. It is particularly important when etching deep features (tens to hundreds of microns) such as MEMS structures or through silicon vias (TSVs) in silicon, where long etch process times are required, for an optimal balance to be achieved between high etch rates and across wafer etch uniformity. To form these kinds of deep silicon etch features, the so-called ‘Bosch process’ of cyclic deposition/etch steps are typically used. The Bosch process is well-known in the art, and is described, for example, in U.S. Pat. No. 5,501,893.

Accordingly, a great deal of research has been carried out with the purpose of increasing etch rate without sacrificing etch uniformity. Representative examples in the prior art include US2006/0070703 and U.S. Pat. No. 7,371,332. The overwhelming received wisdom in the field is for plasma etch tools to use a process chamber where the internal diameter of the process chamber is considerably larger than the diameter of the wafer being processed. There is a technical explanation for this approach which also constitutes a received wisdom in the field. More specifically, it is considered beneficial for the process chamber to be of a considerably larger internal diameter than the diameter of the wafer being processed because it is believed that a uniform plasma is more readily achieved in such a system, with losses to the walls of the chamber—which result in non-uniformities in the plasma—occurring well away from the edge of the wafer being processed. FIG. 1 of US2006/0070703 is a representative schematic diagram of a typical prior art single wafer ICP plasma etch system. As depicted in this figure, a cylindrical chamber has a central platen support or electrostatic chuck (ESC) that locates the circular wafer to be processed. A plasma is initiated and sustained by coupling RF power through an antenna, in this case a multi-turn coil, into the gas within the chamber. The gas enters at the top of the chamber and the by-products of the etch process exit the bottom of the chamber using a suitable pumping arrangement. The wafer platen can also be RF driven in certain system configurations in order to provide further control of the incident ions on the wafer surface as is well-known in the art. Although FIG. 1 of US2006/0070703 is schematic, in fact the relative dimensions of the wafer to the chamber are essentially accurate representations of the prior art.

### SUMMARY

The present inventors have realised that when etching a wafer in a standard plasma etching chamber of the type shown in FIG. 1 of US2006/0070703, a proportion of the active etchant gas completely bypasses the wafer by flowing down the sides of the chamber. The present inventors have also recognised that this is in efficient in terms of gas usage. Because pumping occurs below the wafer surface, the majority of the gas introduced into the chamber may never reach the wafer surface. For example, with a chamber of the type shown in FIG. 1 of US2006/0070703 having a 350 mm diameter with a 200 mm diameter wafer in the centre of the chamber, around two thirds of the gas flow would exit directly to the pump. Additionally, the present inventors have realised that this configuration results in higher etch rate near to the centre of the wafer with lower etch rates being observed at the periphery of the wafer. This is represented schematically in FIG. 1, which shows silicon etch rates as a function of position from the centre of the wafer. It can be seen that the prior art configuration promotes a centre high etch rate which essentially follows the gradient of etchant concentration as a function of distance from the centre of the chamber to the periphery of the wafer.

It is also known to provide two chamber plasma etching arrangements in which plasma generated in a first chamber flows into a second, processing, chamber in which the substrate resides. Again, it is received wisdom in the field for the internal diameter of the process chamber to be considerably larger than the diameter of the wafer being processed. Additionally, the present inventors have appreciated that the first chamber in which the plasma is generated is generally of a relatively large size in comparison to the diameter of the wafer. US2007/0158305 and EP2416351 both disclose what are effectively two chamber arrangements in which a guide, which may be a frusto-conical guide, is used to direct plasma towards a substrate located in the second chamber. However, the region of the first chamber in which the plasma is generated is of a diameter substantially greater than the diameter of the wafer being processed.

The present invention, in at least some of its embodiments, addresses one or more of the above described problems. In particular, at least some embodiments of the invention can provide improved etch uniformity and/or improved gas utilisation in comparison to a conventional system. Additionally, at least some embodiments of the invention can provide an improved etch rate in comparison to a conventional system.

For the avoidance of doubt, whenever reference is made herein to ‘comprising’ or ‘including’ and like terms, the invention is also understood to include more limiting terms such as ‘consisting’ and ‘consisting essentially’.

According to a first aspect of the invention there is provided a plasma etching apparatus for plasma etching a substrate, the substrate including:

a first chamber having a plasma generation region, the plasma generation region having a cross-sectional area and shape;

a plasma generation device for generating a plasma in the plasma generation region;

a second chamber into which the plasma generated in the plasma generation chamber can flow, wherein the second chamber defines an interior having a cross-sectional area and shape, and the cross-sectional area of the interior is greater than the cross-sectional area of the plasma generation region;

a third chamber having a substrate support for supporting a substrate of the type having an upper surface to be plasma etched, wherein the third chamber has an interface with the second chamber so that the plasma, or one or more etchant species associated with the plasma, can flow from the second chamber to plasma etch the substrate;

in which:

the inner cross-sectional area and shape of the second chamber interior substantially corresponds to the upper surface of the substrate; and

the substrate support is disposed so that, in use, the substrate is substantially in register with the interior of the second chamber, and the upper surface of the substrate is positioned at a distance of 80 mm or less from the interface.

The substrate to be processed and the interior of the second chamber may each have at least one width. The ratio of the width of the interior of the second chamber to the width of the substrate may be 1.15 or less, 1.1 or less, 1.0 or more, 0.85 or more, 0.9 or more, or any combination of these ratio values. In particular, the ratio of the width of the interior of the second chamber to the width of the substrate may be in the range 1.15 to 0.85, preferably 1.1 to 0.9.

More preferably, the ratio of the width of the interior of the second chamber to the width of the substrate may be in the range 1.15 to 1.0, preferably 1.1 to 1.0.

It will be appreciated that typically the substrate to be processed and the interior of the second chamber are of circular cross-section, in which instance the widths referred to above are diameters. In principle, the substrate to be processed and the interior of the second chamber may be of a different cross-sectional shape, and again in principle such a non-circular shape may have more than one characteristic width. In these embodiments, each characteristic width associated with the cross-sectional shape will have a corresponding ratio of the width of the interior of the second chamber to the width of the substrate. In these embodiments, each width ratio may satisfy the quantitative criteria above.

In some embodiments, the substrate support is disposed so that, in use, the upper surface of the substrate is positioned at a distance of 60 mm or less from the interface.

In some embodiments, the substrate support is disposed so that, in use, the upper surface of the substrate is positioned at a distance of 10 mm or more from the interface.

Preferably, the substrate support is disposed so that, in use, the upper surface of the substrate is positioned at a distance in the range 10-60 mm from the interface.

The plasma generation region and the interior of the second chamber each have a cross-sectional area the ratio of the cross-sectional area of the plasma generation region to the cross-sectional area of the second chamber may be in the range 0.07 to 0.7.

Typically, the first and second chambers are co-axial. In use, the substrate is typically also co-axial with the first and second chambers.

Typically, the first and second chambers are both of circular cross-section. Typically the substrate to be plasma etched is also of circular cross-section.

The first chamber may be of a bell jar shape, i.e., the first chamber may have a non-constant circular cross-section which varies as a function of position along a longitudinal axis of the chamber so as to flare out towards the second chamber.

The apparatus may further include a baffle disposed or near to the substrate support in order to channel a gas flow in the vicinity of substrate. The baffle may be disposed so as to, in use, increase a retention time of etchant species around a periphery of the wafer.

In some embodiments, the interface is defined by a spacer element disposed between the second chamber and the third chamber. The spacer element may be an annular element such as a ring. The use of a spacer element is convenient because it enables the distance between the upper surface of the substrate and the interface to be varied and fine-tuned.

Generally, the second chamber is not equipped with a plasma generation device. Instead, only the first chamber has an associated plasma generation device.

Typically, the substrate support is configured to support a substrate having a diameter of at least 200 mm. Excellent results have been achieved with the present invention when applied to wafers having diameters of 200 mm and 300 mm.

The present invention is readily applicable to a great many etch materials and process gases, including but not limited to Si, GaAs, polymer, Al etch materials and fluorine, chlorine and oxygen based chemistries. The present invention may be applied to etching using the Bosch process of alternate etching and deposition steps.

The plasma generation device may be an ICP (inductively coupled plasma) source, a helion source, an ECR (electron cyclotron resonance) source, or any other convenient device.

Plasma etching apparatus of the invention may be provided as an original item of manufacture. Alternatively, an advantage of the present invention is that it is possible to retrofit existing plasma etching apparatus of the type wherein a processing chamber is provided having a cross-sectional area which is substantially greater than the area of the upper surface of the substrate to be etched.

The plasma etching apparatus may be provided in combination with the substrate, the substrate being supported by the substrate support. However, the invention pertains also the plasma etching apparatus when not in use or prior to use, i.e., without the substrate present on the substrate support.

According to a second aspect of the invention there is provided a kit for retrofitting an existing plasma etching apparatus in order to provide a retrofitted plasma etching apparatus according to the first aspect of the invention. The kit may include:

an adapter for connection to one or more portions of the existing plasma etching apparatus, the adapter including a sleeve which is configured to act as the second chamber when the adapter is connected, and further including connection means permitting the adapter to be connected to said one or more portions of the existing plasma etching apparatus to locate the sleeve in place.

The adapter may include one or more flange portions for connection to one or more portions of the existing plasma etching apparatus. The adapter may include an upper flange portion and a lower flange portion. The flange portions may form part of a structure which includes the sleeve. Alternatively the flange portions may form part of a structure in which the sleeve can be housed. This structure may include an outer sleeve for housing the sleeve which is configured to act as the second chamber.

The kit may further include a spacer element configured to be disposed underneath the sleeve and connectable to the adapter and/or the existing plasma etching apparatus so as to define the interface between the second chamber and the third chamber in the retrofitted plasma processing apparatus.

According to a third aspect of the invention there is provided a method of plasma etching a substrate including the steps of:

i) providing a plasma processing apparatus according to the first aspect of the invention;

ii) causing the substrate to be supported by the substrate support so that the substrate is substantially in register with the interior of the second chamber and the upper surface of the substrate is positioned at a distance of 80 mm or less from the interface;

iii) generating a plasma in the plasma generation region; and

iv) causing the plasma, or one or more etchant species associated with the plasma, to etch the substrate.

Whilst the invention has been described above, it extends to any inventive combination of the features set out above, or in the following description, drawings or claims. For example, any features described in relation with one aspect of the invention is considered to be disclosed also in relation to any other aspect of the invention.

### BRIEF DESCRIPTION OF THE DRAWINGS

Embodiments of apparatus and methods in accordance with the invention will now be described with reference to the accompanying drawings, in which:

FIG. 1 shows silicon etch rate as a function of distance from a silicon wafer centre during etching using a conventional apparatus;

FIG. 2 is a cross-sectional view of an etching apparatus which has been retrofitted to provide a plasma etching apparatus of the invention;

FIG. 3 is a perspective view of the interface region between the second and third chambers of the plasma etching apparatus of FIG. 2;

FIG. 4 shows etch depth as a function of position on a 300 mm silicon wafer;

FIG. 5 shows silicon etch rate as a function of position on a 200 mm silicon wafer;

FIG. 6 shows silicon etch rate and etch depth uniformity as a function of the diameter of the second chamber when etching 200 mm silicon wafers; and

FIG. 7 shows silicon etch rate and etch depth uniformity as a function of the gap between the second chamber and the wafer upper surface when etching 200 mm diameter silicon wafers.

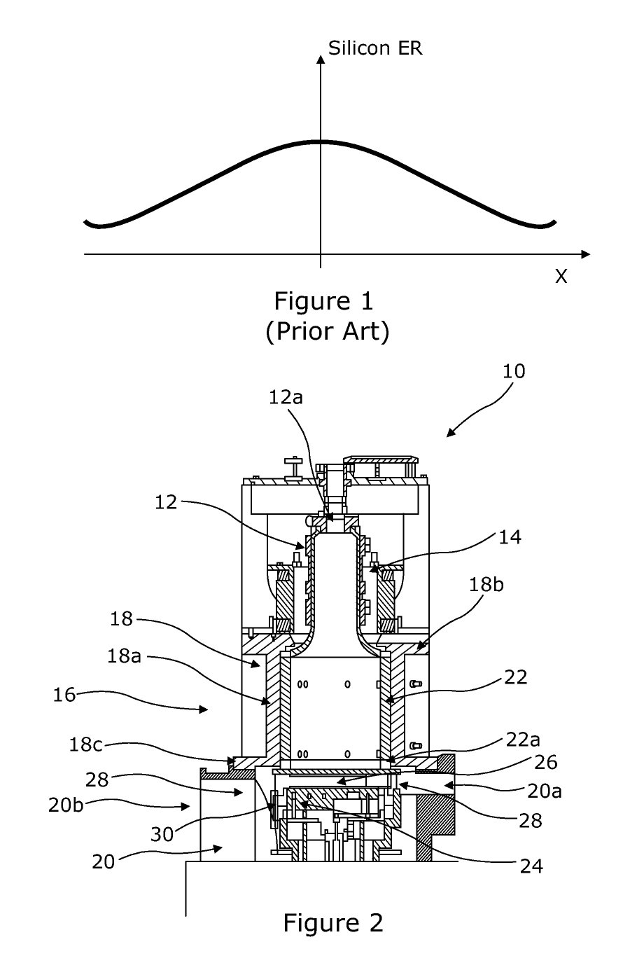

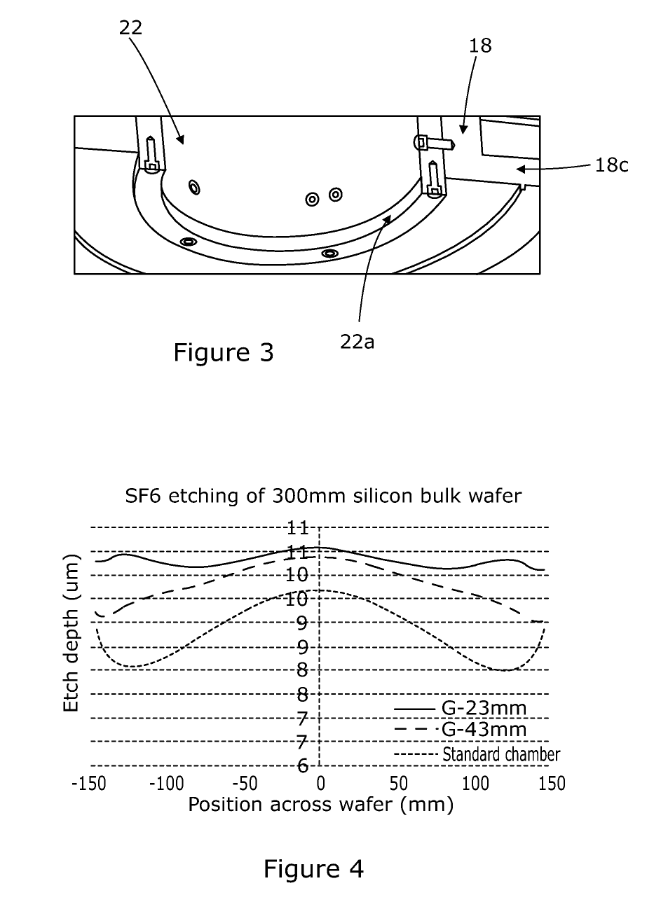

### DETAILED DESCRIPTION OF EMBODIMENTS

FIG. 2 depicts plasma etching apparatus, shown generally at 10, of the invention. The embodiment shown in FIG. 2 is in fact a commercially available plasma etching apparatus which has been retrofitted to produce apparatus in accordance with the present invention. More specifically, the apparatus shown in FIG. 2 is a retrofit of plasma etching apparatus produced by the applicants and marketed under the trade name DSi. The apparatus 10 comprises a first chamber 12 in the form of a ceramic bell jar having a gas inlet 12a through which gases are introduced in order to produce plasma. A portion of the first chamber 12 is surrounded by an ICP source 14 which is used to initiate and sustain a plasma in at least a plasma generation region of the first chamber 12 in a manner which is well-known to those skilled in the art. The lower end of the first chamber 12 flares out into an intermediate portion of the apparatus 10. The intermediate portion is depicted generally at 16 in FIG. 2, and is the main retrofitted component in the apparatus 10. The intermediate portion 16 includes an adapter structure 18 having a sleeve 18a, upper flange portion 18b and lower flange portion 18c. The upper flange portion is connected to the first chamber 12 and other upper portions of the apparatus 10. The lower flange portion is connected to a third chamber 20. The sleeve 18a is sized to carry a reduced-diameter second chamber 22 in the form of a sleeve which is positioned and located within the sleeve 18a. The second chamber 22 can further comprise a lower ring 22a which in this embodiment is connected to the sleeve 22. Below the intermediate section including the second chamber there is the third chamber 20 which houses an electrostatic chuck (ESC) 24 for supporting a wafer 26 to be processed. The third chamber 20 includes a slot valve 20a for introducing the wafer 26 to the apparatus 10 and for removing same. The third chamber further includes an outlet 20b. Gases exit the apparatus from the outlet 20b using a suitable pumping arrangement (not shown) as is well-known to the skilled reader. It is noted that FIG. 2 does not show a complete view of the third chamber. Instead, FIG. 2 only shows an upper portion of the third chamber. The internal diameter of the third chamber 20 is of necessity considerably larger than the diameter of the wafer in order to enable the wafer to be introduced and removed from the apparatus 10. A cylindrical cover 28 is disposed around an upper portion of the apparatus 10 including the first and second chambers 12, 22 for safety purposes.

A baffle 28 is provided around the ESC 24 and a wafer 26 in order to increase the retention time of the etchant gas around the periphery of the wafer 26. A wafer edge protection (WEP) arrangement 30 is also provided.

In the conventional DSi apparatus, a different cylindrical structure serves as the second chamber, and its internal diameter is significantly greater than the diameter of the wafer. In the present invention, the internal diameter of the sleeve 22 (and the ring 22a) are matched to the diameter of the wafer to be processed. In a representative example, the wafer is a 200 mm diameter and the internal diameter of the sleeve 22 and ring 22a is also 200 mm. As described elsewhere herein, it is not mandatory that these diameters should correspond exactly, although advantageous results have been achieved with such an exact matching of the diameters. It will be appreciated that when the wafer 26 is mounted on the ESC 24, the wafer 26 is in register with the second chamber 22.

Examples of improved etching are now described using the apparatus shown in FIG. 2. Etching was performed in accordance with the Bosch process.

In FIG. 4 we show the improvements in process performance achieved in etch rate and uniformity for a Si etch process on 300 mm diameter wafers using a SF₆ chemistry. By reducing the size (ID) of the second chamber from the standard 350 mm to 300 mm while maintaining a chamber to wafer gap of 43 and 23 mm, etch rates increase to 9.8 and 10.3 mm/min, respectively, while uniformity is also significantly improved over the standard value of 9.7%. The results are summarized in Table 1.

TABLE 1
Silicon ER (microns/min) and uniformity values for 300
mm silicon bulk wafer etched with a SF₆ plasma; standard
(350 mm ID) and reduced diameter (G = 300 mm ID).

| | Etch rate | Uniformity |
|---|---|---|
| Gap | [μm/min] | [±%] |
| G-23 mm | 10.3 | 2.6 |
| G-43 mm | 9.8 | 7.4 |
| Standard chamber | 8.8 | 9.7 |

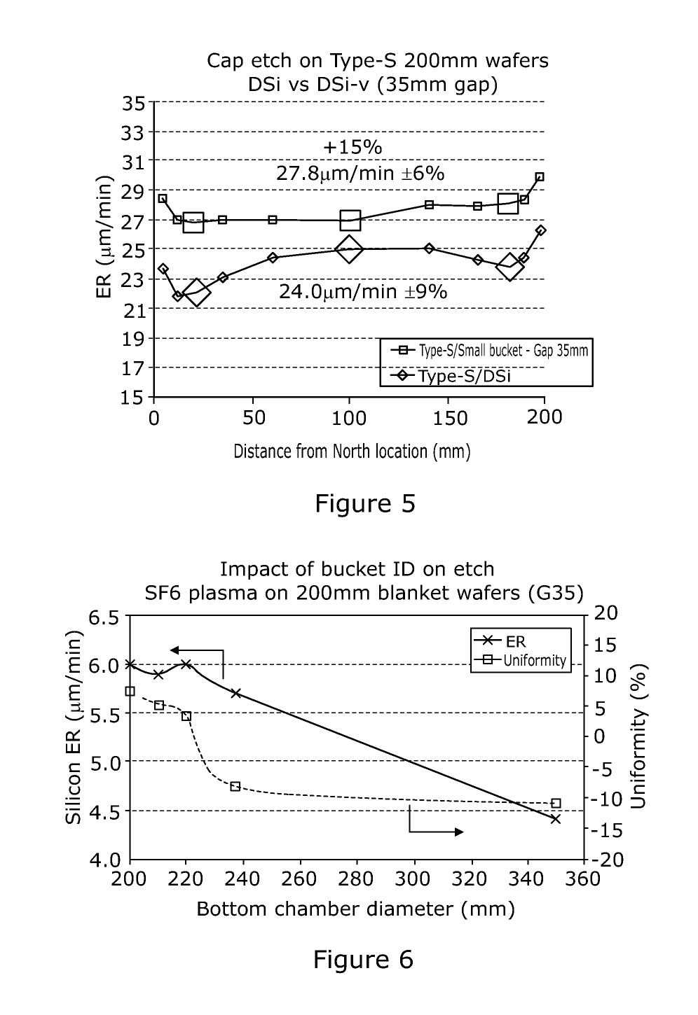

In FIGS. 5, 6 and 7 we can see representative results for 200 mm diameter wafers with a second chamber ID of ˜200 mm. A substantial improvement is seen in all cases when the 200 mm second ID chamber (with a 35 mm gap between the second chamber and the wafer) is compared with the standard 350 mm ID second chamber.

In FIG. 5 we can see a 15% improvement in etch rate for a Bosch Si etch process on patterned Si wafers between the standard chamber and the reduced diameter second chamber. Uniformity is also improved from +/−9% with the standard chamber to +/−6% with the smaller second chamber of the invention.

In FIG. 6 we can see the Si etch rate and uniformity for 200 mm diameter Si wafers as a function of the second chamber internal diameter with a fixed gap between the second chamber and the wafer of 35 mm. At ˜220-235 mm there is a large reduction in uniformity coupled with a more gradual decrease in etch rate as one moves towards larger second chamber IDS.

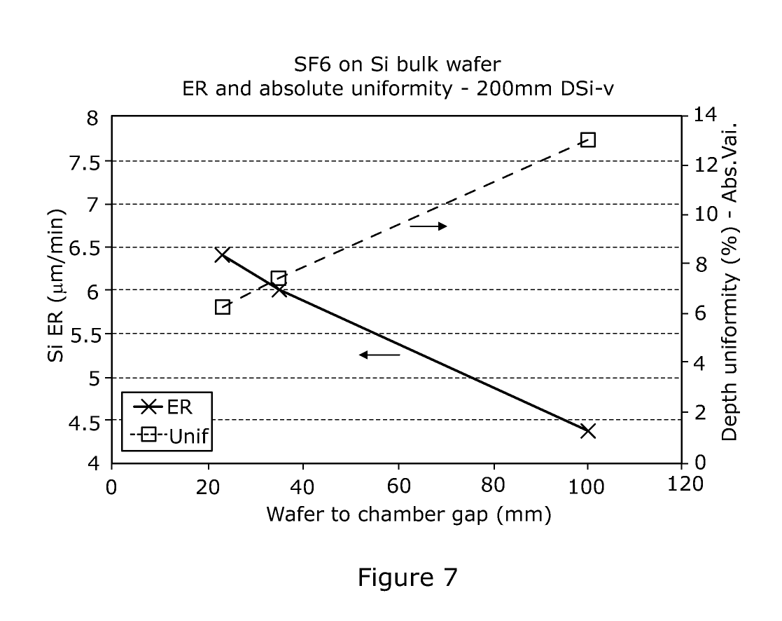

The importance of close coupling of the small lower chamber with the wafer is established in FIG. 7 where a Si etch process is conducted on 200 mm diameter wafers over a range (23-100 mm) of second chamber to wafer gaps. The optimum values for etch rate and uniformity are with the smallest gaps.

Without wishing to be bound by any particular theory or conjecture, it is believed that the advantageous properties described herein can be attributed to the combination of three factors. Firstly, the cross-sectional area of the interior of the second chamber is greater than the cross-sectional area of the first chamber, at least in the region where the plasma is generated. In this way, the volume in which the plasma is initially generated is not too large, and a relatively uniform initial plasma can be formed. In contrast, relatively large plasma generation chambers can give rise toroidally distributed plasmas. It is believed that if the initially generated plasma is not very uniform, then it is at best difficult to provide subsequent processing steps which result in uniform etching. Secondly, the diameter of the second chamber should be close to the diameter of the wafer. This is surprising, since it goes against the received wisdom in the art. In the unlikely but theoretical event that the wafer is not of circular cross-section, then the second chamber should be of a similar shape which closely matches the characteristic dimensions of the wafer. Thirdly, the gap between the wafer (in its in-use position during etching) and the closely matching second chamber should be small.

The apparatus provided by the invention can improve the gas and plasma containment above the plane of the wafer compared to prior art chambers of larger ID. The present invention can avoid or at least reduce the loss of etchant gas going directly to the pumping line, increase the etching rate, and/or improve the cross-wafer depth uniformity. Again, without wishing to be bound by any particular theory or conjecture, it is believed that the present invention can force the etchant gas to interact with the wafer around the wafer periphery before being pumped away. In practice, a balance should be found between this mixing and the reduced conductance that can be caused for pumping the etch products away from the wafer. A baffle might be provided around or in close proximity to the wafer to assist in this regard. However, the use of a baffle is not an essential feature of the invention. The skilled reader will realise that the invention can be implemented and optimised in many different ways, and such variations are within the scope of the invention. For example, it is not necessary that the wafer is supported by an ESC, or that a WEP arrangement is used. Also, instead of retrofitting an existing apparatus, it is possible to produce a new plasma etching apparatus in accordance with the invention. The third chamber may be pumped from a port located at the bottom of the chamber, instead of the side of the chamber. Other plasma generation devices might be contemplated.

What is claimed is:

1. A plasma etching apparatus for plasma etching a substrate, the apparatus including:
a first chamber having a plasma generation region, the plasma generation region having a cross-sectional area and shape;
a plasma generation device for generating a plasma in the plasma generation region;
a second chamber into which the plasma generated in the plasma generation chamber can flow, wherein the second chamber defines an interior having a cross-sectional area and shape, and the cross-sectional area of the interior is greater than the cross-sectional area of the plasma generation region;
a third chamber having a substrate support for supporting a substrate of the type having an upper surface to be plasma etched, wherein the third chamber has an interface with the second chamber so that the plasma, or one or more etchant species associated with the plasma, can flow from the second chamber to etch the substrate;
in which:
the inner cross-sectional area and shape of the second chamber interior substantially corresponds to the upper surface of the substrate; and
the substrate support is disposed so that, in use, the substrate is substantially in register with the interior of the second chamber, and the upper surface of the substrate is positioned at a distance of 80 mm or less from the interface.

2. A plasma etching apparatus according to claim 1 in which the substrate to be etched and the interior of the second chamber each have at least one width, and the ratio of the width of the interior of the second chamber to the width of the substrate is in the range 1.15 to 0.85, preferably 1.1 to 0.9.

3. A plasma etching apparatus according to claim 2 in which the ratio of the width of the interior of the second chamber to the width of the substrate is in the range 1.5 to 1.0, preferably 1.1 to 1.0.

4. A plasma etching apparatus according claim 1 in which the substrate support is disposed so that, in use, the upper surface of the substrate is positioned at a distance of 60 mm or less from the interface.

5. A plasma etching apparatus according to claim 1 in which the substrate support is disposed so that, in use, the upper surface of the substrate is positioned at a distance of 10 mm or more from the interface.

6. A plasma etching apparatus according to claim 1 in which the ratio of the cross-sectional area of the plasma generation region to the cross-sectional area of the second chamber is in the range 0.07 to 0.7.

7. A plasma etching apparatus according to claim 1 in which the first and second chambers are co-axial.

8. A plasma etching apparatus according to claim 1 in which the first and second chambers are both of circular cross-section.

9. A plasma etching apparatus according to claim 1 in which the first chamber is of a bell jar shape.

10. A plasma etching apparatus according to claim 1 in which the interface is defined by a spacer element disposed between the second chamber and the third chamber.

11. A plasma etching apparatus according to claim 1 further including a baffle disposed on or near to the substrate support in order to channel a gas flow in the vicinity of the substrate.

12. A plasma etching apparatus according to claim 1 in which the substrate support is configured to support a substrate having a diameter of at least 200 mm.

13. A plasma etching apparatus according to claim 1 in combination with the substrate, the substrate being supported by the substrate support.

14. A kit for retrofitting an existing plasma etching apparatus in order to provide a retrofitted plasma etching apparatus according to claim 1, the kit including:
an adapter for connection to one or more portions of the existing plasma etching apparatus, the adapter including a sleeve which is configured to act as the second chamber when the adapter is connected, and further including connection means permitting the adapter to be connected to said one or more portions of the existing plasma etching apparatus to locate the sleeve in place.

15. A kit according to claim 14 in which the adapter further includes one or more flange portions for connection to one or more portions of the existing plasma etching apparatus.

16. A kit according to claim 14 further including a spacer element configured to be disposed underneath the sleeve and connectable to the adapter and/or the existing plasma etching apparatus so as to define the interface between the second chamber and the third chamber in the retrofitted plasma etching apparatus.

17. A method of plasma etching a substrate including the steps of:
i. providing a plasma etching apparatus according to claim 1;
ii. causing the substrate to be supported by the substrate support so that the substrate is substantially in register with the interior of the second chamber and the upper surface of the substrate is positioned at a distance of 80 mm or less from the interface;
iii. generating a plasma in the plasma generation region; and
iv. causing the plasma, or one or more etchant species associated with the plasma, to etch the substrate.
